(19) United States
(12) Patent Application Publication
(10) Pub. No.: US 2005/0100555 A1
(43) Pub. Date: May 12, 2005

(54) Homing peptides

(75) Inventors: Constantino Pitzalis, London (GB); Gabriel Stavros Panayi, London (GB)

Correspondence Address: BANNER & WITCOFF, 1001 G STREET N W, SUITE 1100, WASHINGTON, DC, 20001, US

(73) Assignee: KING'S COLLEGE LONDON An Institute Incorporated by Royal Charter of Stra, London (GB)

(21) Appl. No.: 10/488,779

(22) PCT Filed: Sep. 4, 2002
(86) PCT No.: PCT/GB02/04017

(30) Foreign Application Priority Data
Sep. 5, 2001 (GB) 0121499.8
Aug. 1, 2002 (GB) 0127894.5

(51) Int. Cl. A61K 39/00; C12P 21/04; C07K 14/47

(52) U.S. Cl. 424/192.1; 530/350; 435/69.7; 435/320.1; 435/325

(57) ABSTRACT

A synovial tissue binding peptide comprises an amino acid sequence motif comprising RLP, SPS, HSS, LSS, TWS, YSS, NQR, DRL or DHR. Preferred motifs comprise HPRLPFA, APNWRLP, SPSPFRA, SPSRFDQ, VSPSRTT, PLSSAQR, TWSATST, THSSATQ, HTHSSNL, PNHSSPH, ADHSSRH, SDYSSRS, QTHNQRY, TNQRLAI, KSTHDRL, PFHDRHS, HPSDRLS or DRLNHQE. Also provided is a method for the identification of peptides capable of binding to a tissue originating from a first mammalian species, the method comprising the steps of: grafting the tissue originating from the first mammalian species into a subject of a second mammalian species having an attenuated immunological response; introducing a plurality of peptides into the second species; and determining the localisation of the peptides within the second species.

This invention relates to delivery systems targeted to human tissues and more particularly to peptides for use in site-specific delivery and methods for the identification thereof.

Treatment of many conditions where the disease process principally localises to specific organs is unsatisfactory as non-specific systemic therapies are used. Such conditions include rheumatoid arthritis, psoriasis, inflammatory bowel disease and other conditions involving degenerative or inflammatory pathologies. None of the treatments generally employed for these conditions is curative. In addition, the treatments are often bedevilled by side effects. A considerable improvement on current therapies would be represented by the possibility to deliver these drugs directly to the site of disease.

The microvascular endothelium (MVE) plays a major role in the pathogenesis of rheumatoid arthritis (RA) making it an important therapeutic target. RA is a condition characterised by a proliferative synovitis responsible for cartilage and bone damage that leads to progressive joint destruction (1, 2). Florid sprouting of new blood vessels (neo-angiogenesis) is typically seen in the early phases of the RA synovitis suggesting that it is a critical element in this pathological context (3). In the established chronic phase of the disease the MVE is also important as it functions as a conduit for the continuous influx of inflammatory cells from the bloodstream into the joint (4, 5). The extravasation process is a complex phenomenon regulated by a series of integrated adhesion and signalling events that include the interaction of surface cell adhesion molecules (CAMs) and chemokines (CK) (6, 7). In addition to the general mechanisms applicable to all leucocyte types, there is evidence that the specific pairing of ‘homing receptors’ and ‘vascular addressing’, expressed on the surface of migrating lymphocytes and on MVE of different organs respectively, contributes to the selective recruitment of different leucocyte populations to various tissues (8, 9). Well characterised examples include the preferential interaction of L-selectin with GlyCAM and α₄β₇ with MdCAM-1, that facilitate lymphocyte migration to peripheral lymph nodes and intestinal sites, respectively (10-13). Furthermore, pairs of CK and CK-receptors (TARC-CCR4 and TECK-CCR9) appear to co-ordinate ‘homing’ CAMs (CLA and α₄β₇) in facilitating lymphocyte migration to skin and gut tissue, respectively (14, 15). So far, in addition to lymphoid tissues, preferential circulatory pathways have been postulated for the gut, the lung, the skin and the joints (16, 17).

However, the identification of a specific MVE ‘addressin’ for the joints has proven elusive. The reason for this is related, at least in part, to the difficulties of isolating pure populations of synovial endothelial cells. In addition, and more importantly, it is known that culturing isolated MVE cells in vitro causes de-differentiation with loss of important tissue-specific traits such as tight junctions in brain MVE or loss of MAdCAM-1 expression by intestinal MVE (18-21). Thus, targeting the MVE in its own microenvironment is likely to be necessary to identify organ-specific ligands. The development of phage display of random peptides (22-24) and antibody fragment libraries (25-27) has allowed the precise targeting of the MVE of various tissues in vivo in animals. In particular, Ruoslahti and colleagues have succeeded in generating at least one specific homing peptide sequence for each of the seven different organs probed in mice, as well as for tumour vasculature (28, 29). In addition, it was shown that specific homing peptides could be used as targeting devices to concentrate drugs to various tissues in in vivo models (29-31). A similar approach has been used to isolate single-chain variable region (sFv) antibodies from phage display (sFv-PDL) specific for murine thymic endothelium in vivo (32). Lastly, a phage displaying a constrained cyclic RGD peptide that binds to αbβ₃ and αvβ₅ (covalently linked to a 14-amino-acid pro-apoptotic peptide) was shown to home to inflamed synovium and to suppress collagen-induced arthritis (33).

The application of such technology to humans has been prevented because of the technical difficulties and ethical considerations of performing screening studies in vivo. However, it is possible to transplant human tissues into severe combined immunodeficient (SCID) mice. The inventors have successfully adapted this model for the transplantation of human synovium, skin, lymphoid and foetal gut tissues (34-36). Transplants remain viable, becoming vascularised by mouse subdermal vessels that anastomose with the graft human vasculature. The anastomoses are patent and functional as shown by the capacity to deliver antibodies and human cells to the grafts via the mouse systemic circulation (34). Most importantly, the graft MVE maintains the expression of human adhesion molecules forming, in proximity of the anastomoses, transitional areas expressing human and murine CAMs next to each other, that can be up-regulated following intra-graft injection of cytokines (34). Finally, the MVE of the grafts remains within its normal microenvironment, a fact that is likely to facilitate the maintenance of the tissue-specific vascular traits.

None of the prior art referred to above contemplates a peptide screening method suitable for identifying peptides capable of targeting human tissues. One of the objects of the present invention is the provision of such a method and the products thereof.

Accordingly, one aspect of the present invention provides a synovial tissue binding peptide comprising an amino acid sequence motif comprising RLP, SPS, HSS, LSS, TWS, YSS, NQR, DRL or DRH.

The term ‘synovial tissue binding peptide' as used herein refers to a peptide which is capable of specific binding and preferential localisation to synovial tissue following systemic administration. The term ‘motif’, as used herein, refers to a part of a peptide, definable in terms of a series of amino acids, capable of conferring functional, particularly binding specificity, properties on that peptide. Throughout this specification the standard one-letter system of notation for amino acids is used.

The motif may comprise SPSRF. Alternatively, the motif may comprise (T or D)HSS(A or R)(T or H). As a further alternative the motif may comprise HDRL. Preferably, the motif comprises HPRLPFA, APNWRLP, SPSPFRA, SPSRFDQ, VSPSRTT, PLSSAQR, TWSATST, THSSATQ, HTHSSNL, PNHSSPH, ADHSSRH, SDYSSRS, QTHNQRY, TNQRLAI, KSTHDRL, PFHDRHS, HPSDRLS or DRLNHQF.

Peptides according to the present invention are preferably between 3 and 1000 amino acids in length. More preferably, the peptides are between 3 and 100 amino acids in length. Most preferably, the peptides are between 3 and 20 amino acids in length. The motifs of the peptides and/or the remainder of the peptides may contain chemically-modified amino acids, provided that any modifications to the motif do not affect its functional characteristics. Functional homologues of the peptides are also to be regarded as within the scope of the invention. The term “functional homologue” refers to a peptide which retains the synovial tissue binding activity of the peptide on which the homologue is based and which preferably has a motif with a sequence homology of at least 60%, more preferably at least 80%, even more preferably at least 90% and most preferably 95% when compared with the motif of the peptide on which the homologue is based. Amino acid changes between functional homologues are preferably conservative, i.e. involving the replacement of one amino acid with one from a family of amino acids which are related in their side chains.

The peptide may be linear or may be cyclised. When the peptide is linear, it may contain one or more sulphur-containing amino acids at one or both ends of the motif. The sulphur-containing amino acids may be C or M.

The peptide is preferably cyclised.

In certain embodiments, the peptide includes a pair of amino acids capable of facilitating intramolecular cyclisation of the peptide. The members of the pair are preferably located towards opposite ends of the motif and are more preferably located at opposite ends of the motif. The cyclisation of the peptide facilitated by the pair of amino acids may involve only part of the peptide or may involve the whole peptide. Preferably, the whole motif and, more preferably, the whole peptide is cyclised. The pair is preferably C and C, C and M or M and M. In a preferred embodiment, the motif is C-HPRLPFA-C, C-APNWRLP-C, C-SPSPFRA-C, C-SPSRFDQ-C, C-VSPSRTT-C, C-PLSSAQR-C, C-TWSATST-C, C-THSSATQ-C, C-HTHSSNL-C, C-PNHSSPH-C, C-ADHSSRH-C, C-SDYSSRS-C, C-QTHNQRY-C, C-TNQRLAI-C, C-KSTHDRL-C, C-PFHDRHS-C, C-HPSDRLS-C or C-DRLNHQF-C, wherein C- and -C independently represent any type or number of amino acids preceding or following, respectively, the amino acids within the flanking cysteines. Preferably, the number of preceding or following amino acids is less than twenty in each case. More preferably, the number is zero. In a particularly preferred embodiment, the motif is C-KSTHDRL-C.

The synovial tissue binding peptide may consist of any one of the amino acid sequence motifs listed above.

The peptide may be coupled to a pharmacological or diagnostic agent. The pharmacological agent is preferably an anti-inflammatory, cytostatic, cytotoxic or immunosuppressive compound. Alternatively, the pharmacological agent may be a gene encoding a peptide having anti-inflammatory, cytostatic, cytotoxic or immunosuppressive properties. The diagnostic agent is preferably suitable for use in diagnostic imaging. Examples of such agents include radio-opaque dyes, fluorescent dyes and radionuclides.

The pharmacological or diagnostic agent may be coupled to the peptide by means of a linker group. This linker group is preferably a flexible moiety, as would be appreciated by one skilled in the art, and is preferably composed of a further stretch of amino acids. The linker group is preferably hydrolysable under appropriate conditions such that the agent may be released from the peptide in the region of the synovial target.

The peptides of the present invention are capable of preferential localisation to synovial tissue. These peptides may be used to create site-specific delivery systems for the treatment of diseases, e.g. rheumatic diseases, with a prevalent synovial joint involvement. The peptides may be prepared using standard solution-phase or solid-phase peptide synthesis techniques and can be couple to other, e.g. pharmacological or diagnostic, agents for the purpose of site-specific delivery of those agents. Such a strategy allows higher systemic doses of pharmacological or diagnostic agents to be used whilst maintaining a tolerable level of side effects arising from the actions of the agents in tissues other than the synovium.

As a result of their ability to localise to the synovial MVE, the peptides may also have intrinsic therapeutic potential by means of an inhibition of the accumulation of inflammatory cells in the region of the synovium. The effective dose range of the peptides can be easily determined by one skilled in the art using standard techniques. Preferably, the effective dose range may vary from around 0.005mg/kg to around 5 mg/kg body weight, more preferably around 0.5 mg/kg to around 5 mg/kg body weight. Preferably peptide of the present invention is delivered by intravenous administration.

Thus, in another aspect, the invention provides a peptide as described above for use in therapy.

In a further aspect, the invention provides the use of a peptide as described above in the preparation of a medicament for the treatment or prevention of inflammatory and/or degenerative arthropathies.

The invention also provides, in another aspect the use of a peptide as described above in the preparation of a composition for the diagnosis of inflammatory and/or degenerative arthropathies.

The peptide of the present invention may also be used to identify the specific synovial ligand using standard screening techniques. Once the synovial ligand is identified, it may be possible to use it as a therapeutic target.

In yet another aspect, the invention provides a pharmaceutical or diagnostic composition comprising a peptide as described above. The pharmaceutical or diagnostic composition of the present invention comprises any one or more of the peptides of the present invention together with any pharmaceutically acceptable carrier, adjuvant or vehicle. Pharmaceutically acceptable carriers, adjuvants and vehicles that may be used in the pharmaceutical composition of this invention include, but are not limited to, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene- polyoxypropylene-block polymers, polyethylene glycol and wool fat.

The pharmaceutical or diagnostic composition of this invention may be administered orally, parenterally, by inhalation spray, or via an implanted reservoir. Preferably the pharmaceutical or diagnostic composition is administered parenterally by injection. The pharmaceutical or diagnostic composition of this invention may contain any conventional non-toxic pharmaceutically-acceptable carriers, adjuvants or vehicles. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical or diagnostic composition may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical or diagnostic composition of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavouring and/or colouring agents may be added.

The pharmaceutical or diagnostic composition of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The composition, especially when for non-oral administration, is preferably formulated as liposomes. The liposomes are preferably composed of one or more naturally-occurring, preferably neutral, phospholipids such as those found in lecithin. The peptide is preferably present on the exterior surface of the liposomes and used to confer synovial tissue specifically.

The invention also provides, in another aspect, a nucleic acid sequence coding for a peptide as described above. In related aspects, the invention also provides a vector containing such a nucleic acid sequence and a cell transformed with such a vector. Also provided, in a related aspect, is an antibody or fragment thereof capable of binding to the peptide of the present invention.

In still another aspect, the present invention provides a method of identifying peptides capable of binding to a tissue originating from a first animal species, the method comprising the steps of:
i) grafting the tissue originating from the first animal species into a subject of a second animal species having an attenuated immunological response;
ii) introducing a plurality of peptides into the second species; and
iii) determining the localisation of the peptides within the second species.

The first and second species used according to this method are different to each other.

In a preferred embodiment of this method, the peptides are introduced into the second species in the form of fusion proteins with a coat protein of a bacteriophage. The bacteriophage is preferably M13 phage. The coat protein is preferably pIII. Such fusion proteins may be obtained by applying the methodology described in references 22 to 24.

The peptides for use in the method of the present invention may include a pair of amino acids capable of facilitating intramolecular cyclisation of the peptides. The members of the pair are preferably located towards opposite ends of the peptides and are more preferably located at opposite ends of the peptide. The cyclisation of the peptide may involve a part of or the whole peptide. Preferably, the whole peptide is cyclised. The pair is preferably C and C, C and M or M and M. The peptides may be generated by random in vitro synthesis.

In embodiments of the method in which the peptides are introduced in the form of fusion proteins with a bacteriophage coat protein, the peptides are preferably generated by replication of the bacteriophage, nucleic acid sequences encoding the peptides having previously been inserted into the bacteriophage genome. Suitable methodology for generation of peptides in this way can be found in references 22 to 24.

The animal species can be any animal which have a circulation including mammals, birds and reptiles. Preferably the animal species are mammals. Preferably first animal species is a human. The tissue may comprise gut, skin, joint or lymphoid tissue. The tissue preferably comprises synovial tissue. The second species is preferably a rodent and is most preferably a mouse. Preferably, the subject of the second species has severe combined immunodeficiency disease.

The method of the present invention allows the rapid identification of peptides capable of targeting to a specific tissue type. Thus, the method may be used to identify peptides useful for the treatment of, or localisation of pharmacological or diagnostic agents to, a range of conditions where the disease process principally localises to specific organs. Examples of such conditions include psoriasis, inflammatory bowel disease or malignancy. In each of these conditions there is already strong evidence in favour of the expression of tissue specific vascular determinants. The novelty of the method lies in the fact that targets of one species, e.g. human, are identified in living tissues grafted into a second species. This is distinct from the identification of targets by the prior art methods of simple injection of peptides into mice and subsequent organ analysis.

The invention will now be described in more detail by way of example only and with reference to the following figures, of which:

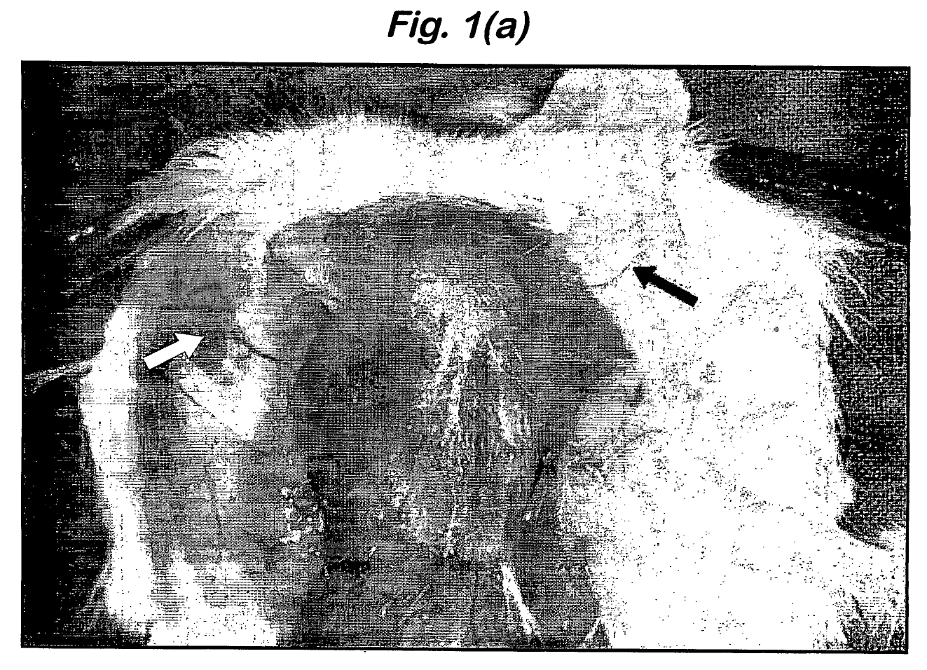

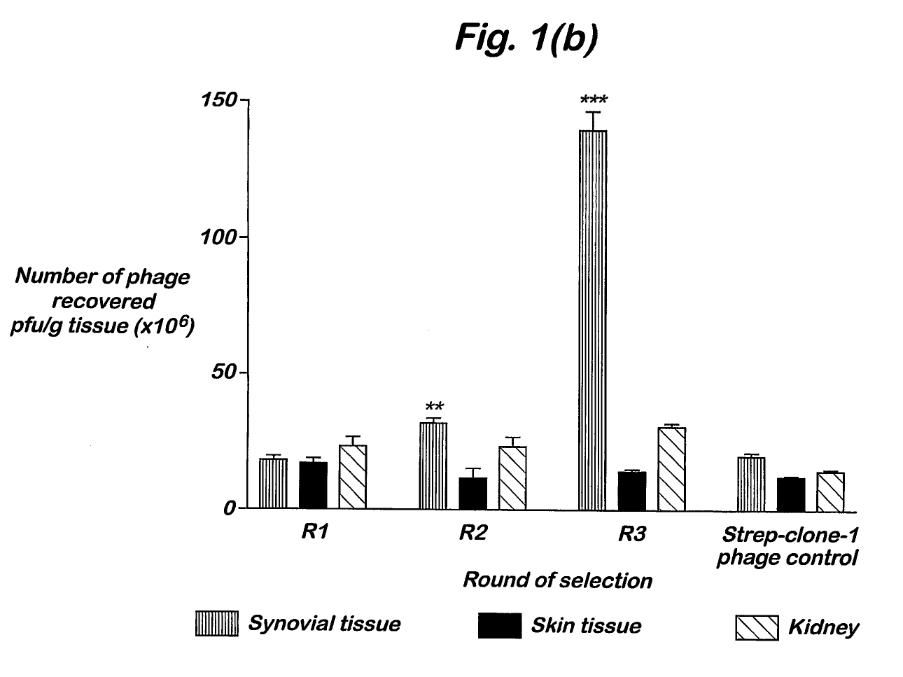

FIG. 1 shows:
a) Macroscopic appearance of human synovial grafts, 4 weeks post-transplantation into SCID mice. The transplants appear healthy and murine blood vessels are clearly visible feeding the graft (arrows);
b) In vivo selection of phage specific for human synovium. The pep-PDL (1×10¹¹ pfu) was injected into the tail vein of SCID mice double transplanted with human synovial and skin tissue (2+2 grafts/animal). 15 minutes after injection the mice were perfused through the heart and phage were rescued from the transplants and the mouse kidney. Phage recovered only from the synovial transplant were amplified and re-injected in two further consecutive rounds of enrichment. Strep-clone-1 phage was used as a negative control. The number of phage (pfu/gram tissue) recovered from 3 consecutive rounds of in vivo selection in double (synovium and skin) transplanted SCID mice is shown. Mouse kidney has been included as murine control tissue. Error bars show standard deviation of the mean from triplicate plate counts from 2 separate experiments (n=2 animals/condition). Differences seen in enrichment rounds, 2 and 3, in the synovial tissue are statistically significant compared to round 1 and strep-clone-1 phage control, ns P=0.48, ** P=0.0004, *** P<0.0001. There are no significant difference in sequential rounds of enrichment in the skin transplants, the mouse kidney and in the strep-clone-1 study, P>0.05 (unpaired, two tailed t-test);
c) In vivo selection of phage specific for human synovium. The pep-PDL (1×10¹¹ pfu) was injected into the tail vein of SCID mice transplanted with human synovium only (2 grafts/animal). The rest of the experimental conditions were identical to b) above except that the in vivo selection cycles were extended to a fourth round. The number of phage (pfu/gram tissue) recovered from each consecutive round is shown. Error bars show standard deviation of the mean from triplicate determinations (n=2 animals/condition). Differences seen in enrichment rounds 3 and 4 are statistically significant compared to round 1, ns P=0.25, ** P=0.0086, *** P<0.0001, whilst no difference was seen in the strep-clone-i control, P=0.055 (unpaired, two tailed t-test);

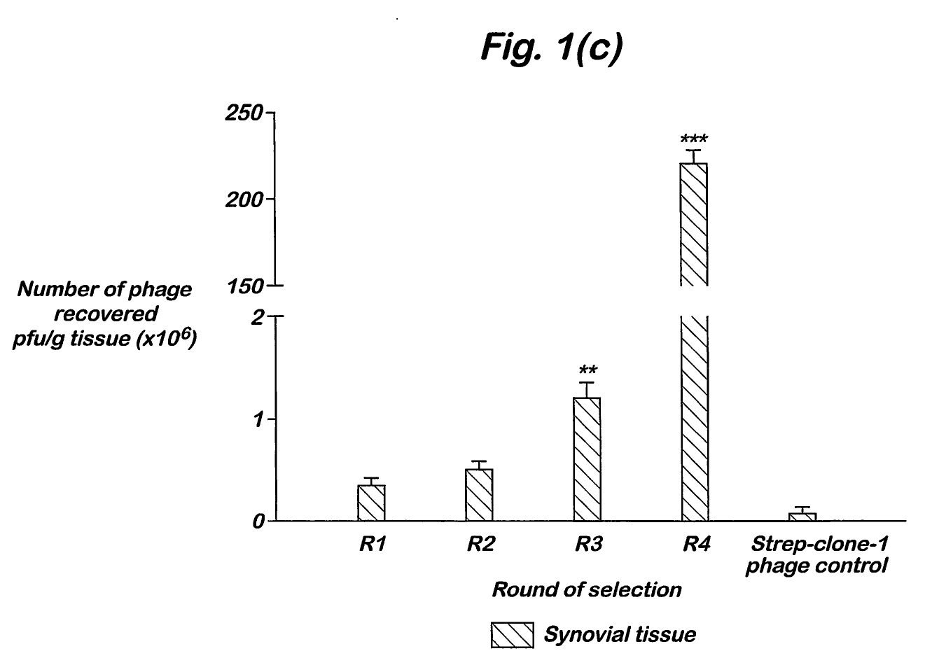

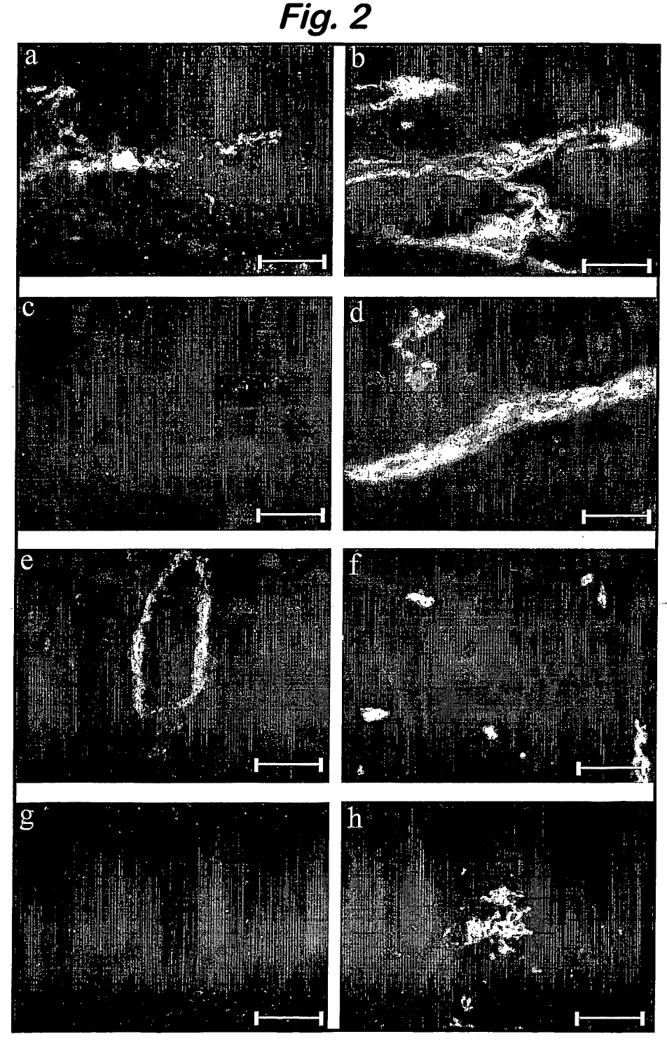

FIG. 2 shows that specific homing phage distinctively localises to synovial graft MVE. The figure shows histological localisation of the peptide phage within the synovial grafts and mouse kidney, detected by immunohistology using anti-M13 coat protein antibody and species-specific vascular markers and visualised by fluorescence microscopy. Representative microscopic fields from the fourth round of selection (frozen tissue aliquots of same samples illustrated in FIG. 1b) are shown. Discrete M13 staining can be clearly seen in (a), while the isotype matched irrelevant antibody showed no staining (c). M13 staining typically co-localises with the human vasculature visualised with anti-human vWf-FITC polyclonal antibody (b and d). However, M13 immunoreactivity (e) shows no co-localisation with murine vasculature within the grafts detected with anti-murine CD31-FITC secondary antibody (f). Likewise, sections of murine kidney taken from the same animal, showed no M13 immunoreactivity (FIG. 2g) in the glomerular capillaries clearly positive for murine CD31 (FIG. 2h). Scale bar=50 μm;

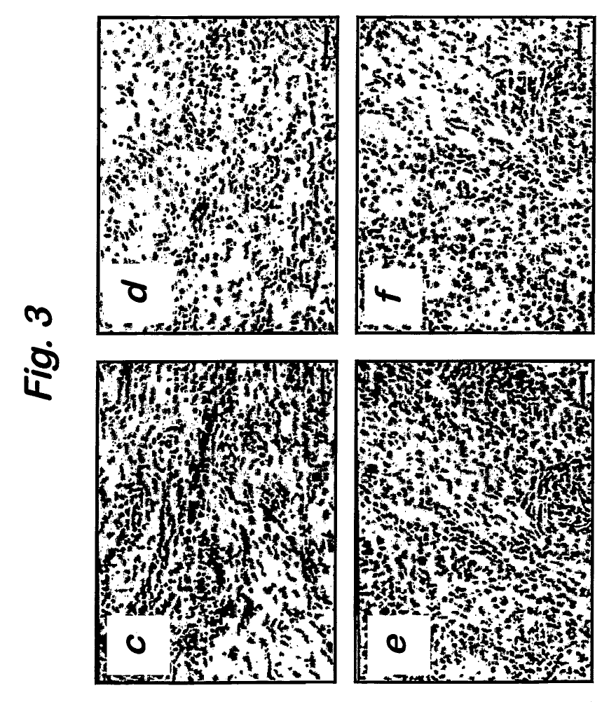

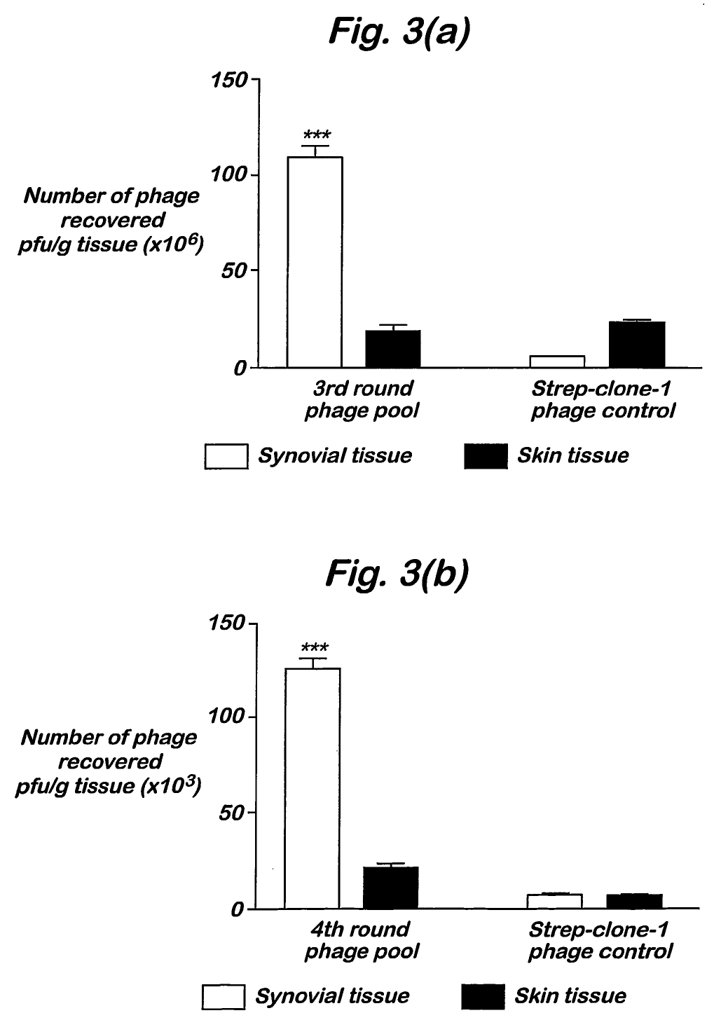

FIG. 3 shows that peptide phage recovered from synovial grafts maintain their tissue homing specificity in vivo in double transplanted animals. In (a), pooled synovial homing peptide phage from the 3^{rd} round of in vivo selection (isolated as illustrated in FIG. 1b) were injected into the tail vein of SCID mice (1×10¹¹ pfu), double transplanted with human skin and synovium obtained from a patient with osteoarthritis (OA). Equal concentrations of strep-clone-1 phage were used as an irrelevant phage control. The number of phage (pfu/gram tissue) recovered from synovium and skin grafts following 15 min. recirculation are shown. Error bars show standard deviation of the mean from triplicate plate counts from duplicate experiments. There is a highly significant statistical difference in the number of phage recovered from synovial grafts compared to skin grafts in the animals injected with synovial homing peptides *** P<0.0002 (unpaired, two tailed t-test). Similar differences were seen when this is compared to the number of strep-clone-1 phage recovered from either synovial or skin grafts. No significant difference is seen in the number of strep-clone-I phage and the synovial homing phage recovered from skin grafts P=0.21 (unpaired, two tailed t-test). In (b), pooled synovial homing phage from the 4^{th} round of in vivo selection (isolated as illustrated in FIG. 1c) were injected (20×10⁸ pfu) into the tail vein of SCID mice, double transplanted as above. The rest of the experimental conditions were also identical to (a). Again specific localisation of the phage to the synovium was seen: *** P<0.0001 (unpaired, two tailed t-test) as described above (a). Frozen graft specimens from the experiments shown in (b) were analysed by immunohistology to illustrate the level of M13 phage localisation to tissue grafts (c-f) It can be observed that there is a considerable M13 staining in the synovial grafts (c) but only minimal M13 immunoreactivity in engrafted skin (e). Controls show only background staining (d and f). Scale bar=50 μm;

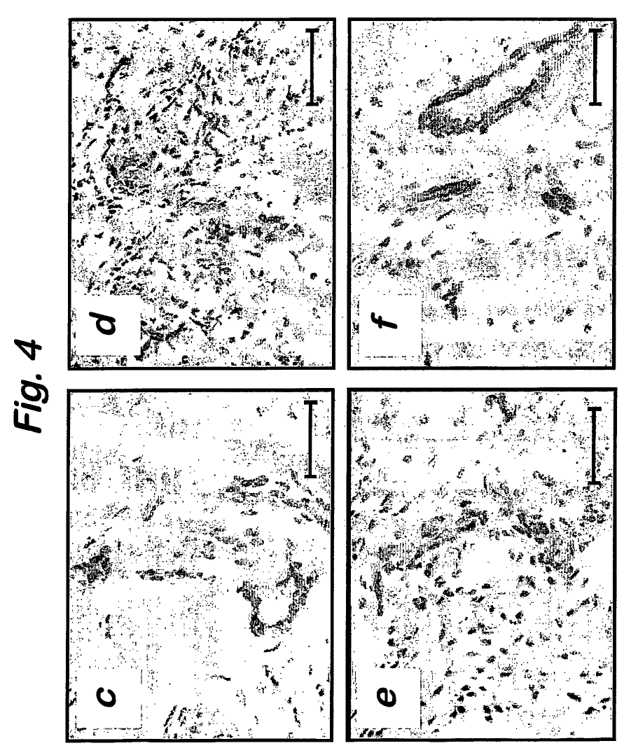

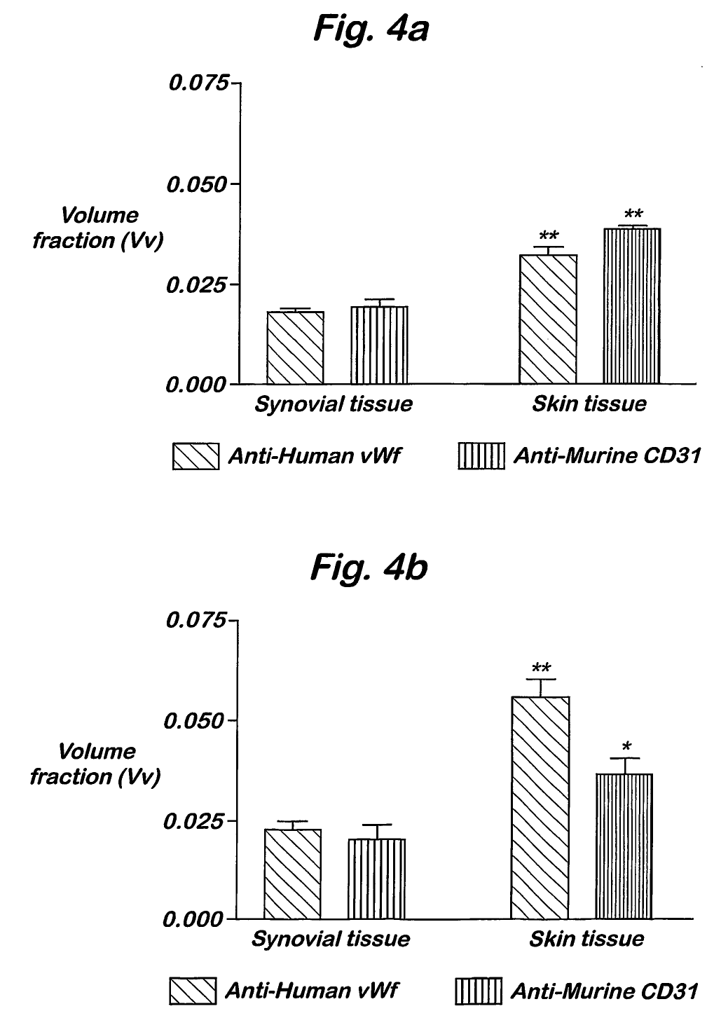

FIG. 4 shows the degree of human and mouse graft vascularity. The degree of vascularisation in frozen tissue aliquots of the samples described in FIGS. (3a) and (3b) was determined by immunohistochemistry by the staining of the human and mouse vascular endothelium using species-specific anti-human vWf and anti-murine CD31 antibodies. The volume fraction (Vv) of immunostained human and murine vessels was determined microscopically using a point counting method as described in the methods. Error bars indicate the standard deviation of the mean from three cutting levels. There is a slight but statistically significant lower endothelial area in the synovial compared to skin grafts in both experiments. This applied to both human vasculature (** P=0.0043 in c and ** P=0.003 in d) and murine (** P=0.00l in c and * P=0.046 in d) vessels (unpaired, two tailed t-test). Representative fields of synovial (c and d) and skin grafts (e and f) stained with anti-human vWf (c and e) and anti-mouse CD31 (d and f) are shown. Scale bar=50 μm;

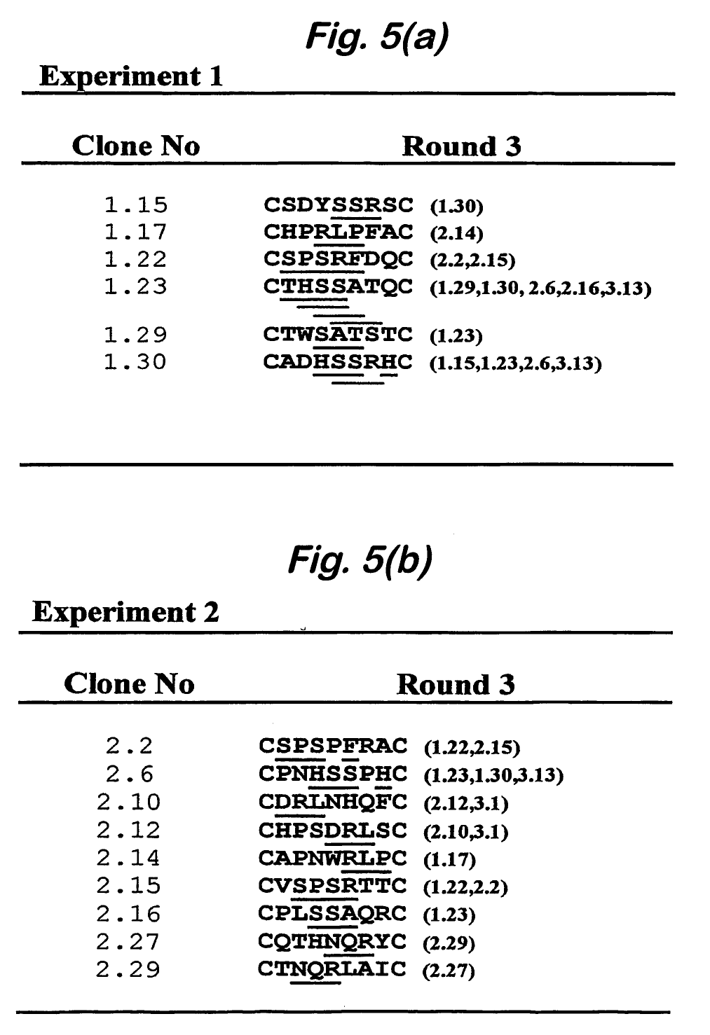

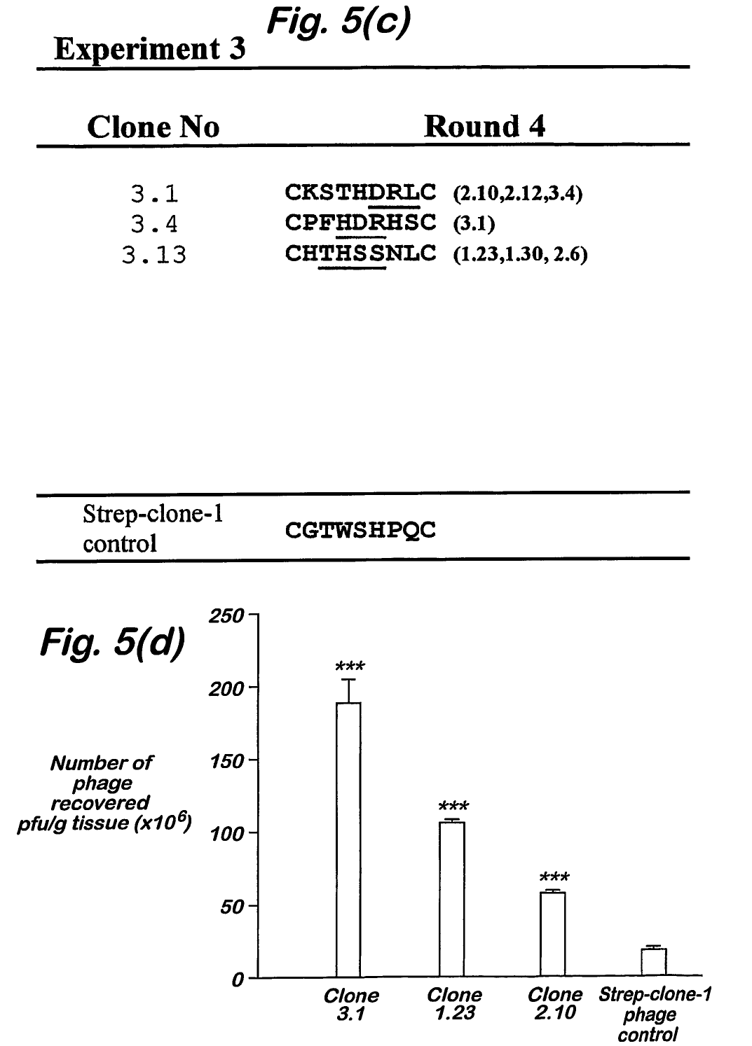

FIG. 5 shows a peptide inserts sequence analysis of synovial homing phage and the in vivo homing properties of phage clones displaying candidate peptides. Peptide inserts, from 30 randomly selected synovial homing phage clones obtained from the final round of in vivo selection of each of the three independent experiments, were sequenced as described in the materials and methods. Alignment of the sequences obtained and multiple comparison within and between experiments identified consensus motifs. For each individual experiment, the complete peptide sequence of those clones displaying consensus motifs are shown (a, b and c). Underlined amino acids indicated candidate motifs with some clones containing multiple overlapping motif regions. In parentheses is shown the occurrence of the same motif in the different clones (see also text). Three individual clones (3.1, 1.23 and 2.10) with a high consensus motif occurrence were amplified and re-injected (1×10¹¹ pfu) into SCID mice transplanted with human synovium (2 grafts/animal). Equal concentrations of strep-clone-1 phage were used as an irrelevant phage control. After 15 minutes recirculation the mice were perfused and the phage concentration in the transplants were determined. Error bars show standard deviation of the mean from triplicate plate readings (n=2 animals/condition). There is a highly significant statistical difference in the number of phage recovered from synovial grafts of animals injected with the candidate phage clones compared to the strep-clone-i phage injected mice *** P<0.0001 (unpaired, two tailed t-test);

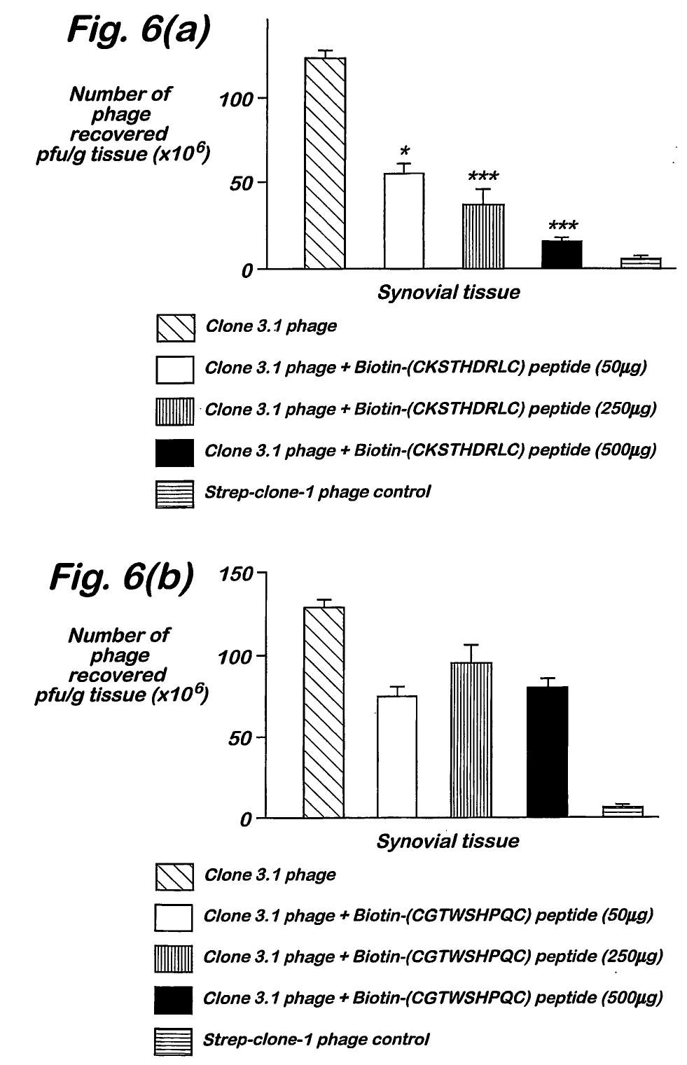

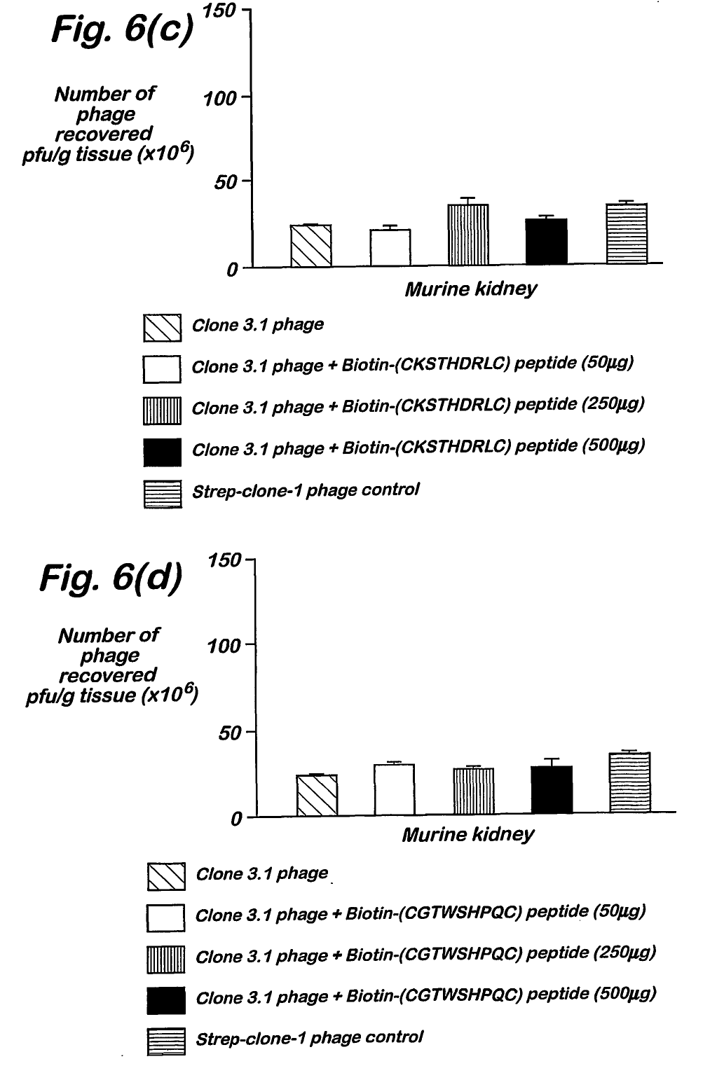

FIG. 6 shows that the synthetic biotinylated peptide CKSTHDRLC localises in vivo specifically to synovial grafts and competes for the cognate tissue ligand with the original peptide phage. SCID mice transplanted with human synovial tissue (2 grafts/animal) were injected intravenously with 1×10¹¹¹ pfu of 3.1 phage clone with and without the biotinylated CKSTHRDLC synthetic peptide (a) at three dose groups (50, 250 and 500 μg/mouse in 200 μL dose volume) or equivalent doses of biotinylated CGTWSHPQC synthetic peptide control (b). Equal concentrations of strep-clone-1 phage were used as an irrelevant phage control. After 15 minutes circulation time, animals were sacrificed and the number of phage in the grafts as well as in the murine kidney (c and d) determined, as described in material and methods. Error bars show standard deviation of the mean from triplicate plate readings (n=3 animals/dose group). It can be seen that the CKSTHDRLC synthetic peptide (a) dramatically inhibits in a dose dependent fashion graft localisation of the parent 3.1 phage clone (over 80% at maximal dose). In contrast, the control peptide has no significant effect on the degree of graft homing of the 3.1 phage clone (b) *** P<0.0001, * P<0.05 (unpaired, two tailed t-test). In addition, no difference was observed between the various groups in the number of phage recovered from murine kidneys. P=0.05 (unpaired, two tailed t-test);

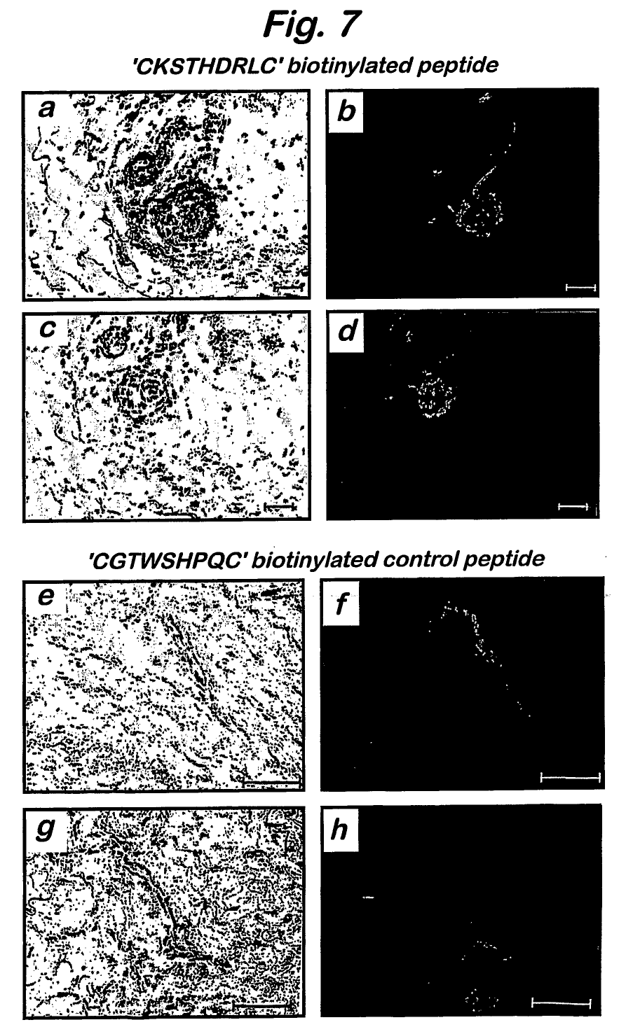

FIG. 7 shows that the histological distribution of biotinylated CKSTHDRLC peptide shows localisation in vivo to human vessels in synovial grafts. Frozen tissue aliquots of the samples described in FIG. 6 were analysed by immunohistology applying an alkaline phosphatase ABC detection system visualised with vector red. Sections were then double stained with anti-human vWf-FITC. Grafts from mice injected with the 3.1 phage clone and 500 μg/mouse biotinylated CKSTHDRLC synthetic peptide clearly show specific immunoreactivity co-localising with human vasculature (a and b). No staining is detected when ABC-AP was omitted from the sequential section (c), although human vWf-FITC positive vessels are present (d). Application of ABC to grafts from mice injected with the 3.1 phage clone and 500 μg biotinylated CGTWSHPQC synthetic control peptide shows no specific immunoreactivity (e) indicating that in the control peptide does not localise to the grafts, despite the presence of blood vessels (f). Again no staining is detected when sequential sections (g), although human vWf-FITC positive vessels are present (h). Scale bar-50 μm.

In these Examples, we report the identification of novel synovial homing peptides isolated from a disulphide-constrained 7 amino acid peptide phage display library (pep-PDL) following several cycles of enrichment in vivo in the human/SCID mouse transplantation model. This is the first time that peptides with homing properties specific for a human synovial MVE have been reported. The use of these peptides to construct targeting devices capable of concentrating therapeutic/diagnostic materials to the synovium may have a considerable impact in the treatment ofjoint diseases.

Materials and Methods

Validation of the peptide phage display library (pep-PDL) in vitro.

Biopanning and sequencing of streptavidin specific peptide phage. The disulfide-constrained (7 amino acids with a flanking cysteine at each end of the peptide) cyclic M13 phage display library (Ph.D.C7C™ system, New England Biolabs, Hitchin, UK) was used throughout this study. The library was validated first by the manufacture's streptavidin/biotin panning technique using the standard reagents provided. After the third round of panning, the DNA from 10 randomly selected phage clones was sequenced using an ABI 377 DNA sequencer after PCR with BigDye™ Terminator Cycle sequencing kit (Applied Biosystems, Warrington, UK) with the primer-96gIII (New England Biolabs).

Selection of synovial homing peptides in vivo in the human/SCID mouse transplantation model.

Human tissue transplantation into SCID animals. Synovial and skin samples were obtained at joint replacement surgery from RA and osteoarthritis (OA) patients after informed consent approved by the Ethics Committee (LREC 98/11/27). Beige SCID C.B-17 mice were singly or double transplanted with synovial and skin tissues subcutaneously as previously described (34).

Selection in vivo of synovial specific phage. Synovial homing phage were isolated by 3-4 cycles of enrichment in SCID mice transplanted with human tissues at 4-6 weeks of age. Four weeks post-transplantation the pep-PDL library (1×10¹¹ pfu in 200 μL saline final volume) was injected into the tail vein of anaesthetized animals. After 15 minutes (in vivo phage circulation time), while under deep/terminal anaesthesia (Sagatal, 5 μg/mouse, Rhone Merieux, France) the mice were perfused via the left ventricle with approximately 50-100 mL of saline to ensure phage clearance from the blood pool. Grafts and various mouse organs were then extracted and divided into two aliquots, weighed and processed as necessary for phage recovery and histological analysis. The aliquot assigned for immunohistology was embedded in Optimal Cutting Temperature compound (OCT, Miles, Calif.), snap frozen in liquid nitrogen cooled isopentane (BDH) and stored at −70° C. until analysis. The aliquot used for phage recovery was washed three times in TBS (150 mM NaCl, 50 nm Tris, pH7.4, Sigma, Poole, UK) then homogenized in 1 ml of TBS containing protease inhibitor cocktail (Sigma). However, the homogenate of the samples showed in FIG. 1b were washed five more time in TBS which lead to a lower recovery of phage. Phage were eluted from the tissues with 1.6 mL of 0.1M glycine, pH 2.0 and after 10 minutes incubation, neutralized with 36 μL of 2M Tris base. To determine number of the phage in the eluate, in each round of selection, tittered triplicate samples of the eluate was added with the E. coli host ER2737 (New England Biolabs) into melted LB agar top (7 g/L agarose, 1 g MgCl₂.6H₂O, Sigma), which were then plated onto IPTG/Xgal LB agar plates (50 mg/L isopropyl β-D-thiogalactoside, 40 mg/L 5-Bromo-4-chloro-3-indolyl-β-D-galactoside, Kramel Biotech, Cramlington, UK). After a 37° C. overnight incubation the peptide phage, appearing as blue plaques, were counted and the yield of phage localising to each individual tissue determined. For synovial grafts only, the remainder of the eluate was amplified by culturing the phage as individual plaques on IPTG/Xgal LB agar plates as described above for tittering. The amplified phage in the plaques were recovered from the agar by homogenizing the agar top layer in LB media, centrifuging and then precipitating the supernatant with PEG/NaCl (3.3% polyethylene glycol 8000/0.4 M NaCl, Sigma). The resultant pool of phage was resuspended in TBS and tittered, as described above, for re-injection in subsequent rounds of in vivo selection. Two or three further cycles of in vivo selection were performed to enrich for synovial specificity.

Sequencing ofpeptide-encoding DNA inserts. The sequence of the DNA inserts encoding for the peptides displayed by the phage homing specifically to the synovial grafts were determined as described above for the validation of the pep-PDL. A sample of 30 phage clones was picked at random, after the last round of in vivo selection and sequenced. Alignment by manual comparison of the sequences was used to identify consensus motifs.

Confirmation of synovial homing specificity in single phage clones displaying consensus motifs. 1×10¹¹ pfu (200 μL saline fmal volume) of three single phage clones (1.23, 2.10 and 3.1) displaying consensus motifs or the strep-clone-1 phage control were and injected intravenously in separate animals transplanted with human synovium as described in ‘Selection in vivo ofsynovial specific phage’. The number of study and control clone phage localising to synovial grafts was determined as described in the same section.

In vitro synthesis of synovial homing peptides. Some of the peptides carrying consensus motifs, identified as above, were synthesized in vitro by Alta Biosciences (Birmingham University, UK) using fMOC chemistry in an automated peptide synthesizer that also allowed the incorporation of a biotin label (37). The peptide was prepared to a purity of >95% by reverse phase chromatography and freeze dried in 2 mg/vial aliquots. Prior to use, the peptide was solubilised in 10 μL of DMSO (BDH) and reconstituted to a final concentration of 4 mg/mL in 0.1M ammonium acetate (pH 6.0, Sigma).

Competitive localisation to human synovial grafts of synthetic biotinylated peptide CKSTHDRLC against the parent 3.1 phage clone. SCID mice were transplanted with human synovial tissue and injected intravenously, as describe above for the in vivo selection experiments, with 1×10¹¹ tpfu of 3.1 phage clone in presence or absence of increasing concentrations of the biotinylated CKSTHRDLC synthetic peptide (50, 250 and 500 μg/mouse in 200 μL dose volume) or the biotinylated CGTWSHPQC synthetic peptide control. Controls also included animals injected with 1×10¹¹ pfu of strep-clone-1 or biotin alone. After 15 minutes circulation time, mice were perfused and the number of phage in the transplants determined as described above. Histological analysis was performed as described below.

Immunohistological Analysis

Assessment of tissue localization of M13-phage. M13 coat phage protein was detected on grafts extracted from animals previously injected either with the whole pep-PDL, pooled phage clones or single phage clones by standard double immunohistochemistry/fluorescence as previously described (38). Briefly, acetone fixed serial cryo-sections (10 μm) were first incubated with anti-M13 Mab (Pharmacia, Uppsala, Sweden) followed by indirect immunoalkaline phosphatase immunohistochemistry (LSAB, Dako, Ely, UK) visualised by Vector Red substrate (Novacastra Labs Ltd, Newcastle upon Tyne, UK) under fluorescence microscopy. Sections were then double stained with either FITC-conjugated sheep anti-human von Willebrand factor (vWf)—Serotec, Kidlington, UK) or rat anti-murine CD31 (clone MEC13.3, Pharmingen, San Diego, Calif.) to stain human and murine vessels within the grafts. A murine anti-Aspergillus Niger glucose oxidase [IgG2a] (Dako, UK) was used as an isotype matched irrelevant antibody. Sections were examined using an Olympus BX-60 fluorescence microscope.

Assessment and quantification of human and murine vasculature within the grafts. To assess the degree of vascularisation of the grafts the human and mouse endothelial surface was determining by immunohistochemistry using the above-mentioned species-specific anti-human vWf and anti-murine CD31 antibodies. The volume fraction (Vv) of immunostained human and murine vessels was determined microscopically using a point counting method as previously described (39). Briefly, 2 immunostained sections per transplant were examined exhaustively using a ×25 objective and rectangular 5×5 eyepiece graticule. The fraction of intersections overlying immunostained vessels was determined for each microscope field and the mean Vv of vessels within the transplants was calculated for murine, human and combined vascularity.

Assessment of graft localization of biotinylated peptides. The graft localisation of the biotinylated CKSTHRDLC synthetic peptide was assessed by using the alkaline phosphatase avidin biotin complex (ABC-AP) detection system (Dako, UK) and visualised by Vector Red substrate (Novacastra, UK).

Statistical Analysis. Results are expressed as mean +95% confidence interval unless otherwise indicated. Non-parametric statistical analyses were performed using the PC analysis package SigmaStat 2.0 (Jandel Scientific). Initially, either the Kruskal Wallis, non-parametric ANOVA, or one way analysis of variance were used. Post-hoc significance testing was carried out using Dunn's multiple comparison tests for non-parametric data, or Dunnett's test for parametric data.

Results

Validation of the phage display library in vitro.

Before proceeding with the in vivo selection using the human/SCID mouse transplantation model, the pep-PDL was validated by in vitro biopanning against streptavidin following the manufacturer recommendations. Sequencing of the peptide-encoding DNA inserts in 10 randomly selected clones demonstrated that all clones showed consensus with the predicted sequences expected for this molecule, G-X-F/Y/W-S/N-H-P-Q, where X indicates any amino acid (data not shown). A clone derived from these experiments (strep-clone-1 phage) displaying the specific C-G-T-W-S-H-P-Q-C peptide was used as a control throughout the study.

In vivo selection of synovial specific homing phage using the human/SCID mouse transplantation model.

Phage with homing properties for human synovium were isolated performing multiple cycles of in vivo selection in the human/SCID mouse transplantation model. In the first set of experiments animals were double transplanted with human RA synovium and skin as control (2+2 grafts/animal) and injected with (1×10¹¹ pfu) of the whole library or the strep-clone-1 control phage. After 15 minutes circulation time, the animals were sacrificed and the number of phage localising to the synovial and skin grafts as well as to murine kidney (control murine tissue) was determined as described in the material and methods. In addition, phage recovered from synovial grafts were amplified to 1×10¹¹ pfu and re-injected into a second and third double transplanted animal. Thus, at each round of selection, the pep-PDL could localise to either human synovial or skin tissue. The results, shown in FIG. 1b, demonstrate a significant increase in the number of phage recovered from the synovial grafts, particularly in the third round. On the contrary, no such enrichment was seen in skin grafts or in the mouse kidneys. Furthermore, the strep-clone-I control phage, showed comparable low levels of localisation in all three tissues. Similar level of enrichment, but to a greater magnitude, was observed when the enrichment cycles were extended to a fourth round using animals transplanted only with human synovium (2 grafts/animal). The results of this second set of experiments are shown in FIG. 1c. Once again a progressive enrichment of phage recovered from the grafts can be seen with a remarkable 600 fold increase in the fourth round compared to the first.

Specific homing phage distinctively localise to synovial microvascular endothelium (MVE).

To determine the anatomical localisation of the phage within the synovial grafts, immunostaining for the M13 coat protein was performed on grafts recovered from animals killed after each round of in vivo selection. In transplants from the first and second round there was modest M13 imrnunoreactivity (data not shown), while grafts from the third and fourth round of selection showed strong staining in and around blood vessels. The vascular localisation was confirmed by double immunofluorescence using antibodies recognising species-specific vascular markers. Representative microscopic fields from the fourth round of selection (tissue aliquots of the same experiments illustrated in FIG. 1c) are shown in FIG. 2. In (a), the characteristic M13 staining can be clearly seen, while the isotype matched irrelevant antibody (c) shows no staining. Of most importance, M13 staining typically co-localises with the human vasculature visualised with anti-human vWf-FITC polyclonal antibody (b and d). In contrast, there is no M13 immunoreactivity (e) co-localisation with invading murine vasculature within the grafts, detected with anti-murine CD31-FITC secondary antibody (f). In addition, synovial homing phage do not bind to murine tissue vasculature as shown by the negative M13 staining (g) in the glomerular capillaries that are clearly positive for murine CD31 (h). Thus, following several cycles of targeting in vivo human synovial grafts, the inventors isolated tissue and species-specific phage that preferentially bind to human synovial but not murine MVE or human skin.

Synovial homing phage maintain their tissue specificity in vivo, independently from the original pathology of synovial grafts (RA vs OA).

To assess whether the synovial homing properties were due to the intrinsic characteristics of the synovium or related to the disease status (e.g. RA vs OA), pooled clones recovered from the last round of in vivo selection (3^{rd} and 4^{th} round of the experiments described above) were tested in recirculation studies in vivo using SCID animals double transplanted with skin and human synovium from a patient with OA. As previously, equivalent amounts of strep-clone-1 phage were injected intravenously into control animals. As shown in FIG. 3a and b the number of phage recovered from synovial grafts was significantly greater than from skin transplants. In contrast, in animals injected with the strep-clone-1 phage the numbers of phage in the skin and the synovial tissues are virtually the same and significantly lower than the number of pooled phage homing to the synovium. Further evidence of the preferential synovial graft localisation was obtained using immunohistochemistry. It can be seen that there is a significant M13 phage staining in the synovial graft (FIG. 3c) but only minimal immunoreactivity in the skin transplant (FIG. 3e) with no staining in the negative controls (FIG. 3d and f). Thus, these experiments confirm that synovial homing phage maintain their tissue specificity independently from the pathological features of the synovium of different patients.

Synovial homing properties are independent from the degree of human or murine vascularisation of the grafts.

In order to exclude the possibility that the preferential localization to synovial transplants was the result of an increase in the vascular beds feeding the grafts, the total endothelial surface area for human and mouse vasculature was determined in both the synovial and skin transplants. As can be seen in FIG. 4a and b, skin grafts show a slight increased endothelial surface compared to synovium. This indicates that the degree of graft vascularisation is not responsible for the preferential localisation of the synovial homing peptide-phage to synovial transplants. Representative immunohistological fields from tissue aliquots of these grafts are shown in FIG. 4c-f. There was a comparable evidence of florid neo-angiogenesis in both types of tissue grafts (data not shown).

Sequence analysis of peptide-encoding DNA inserts of synovial specific phage reveals enrichment in specific consensus motifs responsible for the homing properties. In order to investigate whether specific consensus sequences were enriched in the peptides displayed by phage homing preferentially to synovial grafts, the peptide-encoding DNA inserts from 30 clones (selected at random from the last round of selection of three separate experiments) were sequenced. Alignment of the insert sequences identifies several distinct triple and quadruple peptide consensus motifs (FIGS. 5a, b and c). In some clones, several shared or overlapping triple peptide motifs were seen. For example in clone 1.23 the HSS motif (shared by clone 1.30 and 2.6) overlaps with the SSA and the SAT motif, found in 2.16 and 1.29, respectively. In addition, the DRL (2.10, 2.12 and 3.1), and THSS (1.23 and 3.13) motifs were identified in clones recovered from different experiments both from the third and fourth round of selection.

The peptide sequences recovered from in vivo selection in transplanted human synovial tissue in SCID mice were as follows:

Triple peptide motif family

| | |
|---|---|
| Clone name | Sequence |

| | |
|---|---|
| PC3 2.10 | CDRLNHQFC |
| PC3 2.12 | CHPSDRLSC |
| PC4 3.1 | CKSTHDRLC |
| PC4 1.4 | CPFHDRLSC |
| PC3 2.14 | CAPNWRLPC |
| PC3 1.17 | CHPRLPFAC |
| PC3 2.27 | CQTHNQRYC |
| PC3 2.29 | CTNQRLAIC |
| PC3 1.29 | CTWSATSTC |
| PC3 1.15 | CSDYSSRSC |
| PC3 2.16 | CPLSSAQRC |

Quadruple peptide motif family

| | |
|---|---|
| Clone name | Sequence |

| | |
|---|---|
| PC3 2.15 | CVSPSRTTC |
| PC3 1.22 | CSPSRFDQC |
| PC3 2.2 | CSPSPFRAC |
| PC3 1.23 | CTHSSATQC |
| PC4 1.13 | CHTHSSNLC |
| PC3 2.6 | CPNHSSPHC |
| PC3 1.30 | CADHSSRHC |

The inventors next examined whether individual clones containing some of these consensus motifs maintained the same synovial specificity seen with pooled clones. Three clones (1.23, 2.10 and 3.1), one from each set of experiments, were injected intravenously into SCID mice transplanted with human synovium. The results, shown in FIG. 5d, demonstrate a significantly greater localization of the tested clones to synovial grafts in comparison to the control strep-clone-1 phage. In particular, phage clone 3.1, containing the strongly represented DRL motif, showed an increase of approximately 10 fold over the control. Therefore, this phage clone with its displayed peptide (CKSTHRDLC) was selected for the studies described below.

The synthetic peptide CKSTHDRLC retains synovial homing specificity in vivo and competes for the cognate synovial MVE ligand with the parent phage displaying the same peptide.

In order to establish whether some of the candidate peptides described above retained themselves, independently from the original phage, the fuictional capacity of localising specifically to synovial grafts and to inhibit the localisation of the parent phage, a biotinylated synthetic peptide of the sequence expressed by the phage clone 3.1 (CKSTHRDLC) was made. As an irrelevant control, the peptide displayed by the strep-clone-1 phage sequence (CGTWSHPQC) was also synthesized. Synovial transplanted mice were injected with 1×10¹¹ pfu of 3.1 phage clone pre-incubated with a dose range (50-500 μg/animal) of CKSTHDRLC synthetic peptide based on (28). As a control, the experiment was repeated using the CGTWSHPQC synthetic peptide with the same dose range as above. The number of phage localising to the grafts as well as murine tissues was determined as described in the methods. An additional control included animals injected with lxlOi pfu of strep-clone-1 alone. The results are shown in FIG. 6. It can be seen that the CKSTHDRLC synthetic peptide (a) dramatically inhibits in a dose dependent fashion graft localisation of the parent 3.1 phage clone (over 80% at maximal dose). In contrast, the control peptide has no significant effect on the degree of graft homing of the 3.1 phage clone (b). In addition, the 3.1 phage clone localises to the grafts approximately nine times more than the strep-clone-1 control, confirrning the results of the previous experiment shown in FIG. 5d. Finally, in (c) and (d) the level of phage 3.1 localisation to murine kidney is shown. It can be seen that there is only minimal ‘background’ localisation to this mouse organ and that this is not influenced by the study or control peptide.

The inventors next examined the tissue localisation of the CKSTHDRLC synthetic peptide within synovial grafts. Taking advantage of the fact that both study and control peptides were biotinylated, it was possible to precisely detect them by immunohistochemistry using an alkaline phosphatase-ABC detection system visualised by Vector Red substrate. The results, shown in FIG. 7, clearly demonstrate that the peptide strongly localises in vivo to synovial grafts (a) and that it binds principally to human microvascular endothelium as visualised by double staining using FITC conjugated anti-human vWf (b). In contrast, no specific immunoreactivity is detected in grafts from mice injected with the control peptide (e) although human vessels can be clearly seen (f). Sequential sections not treated with the ABC-AP complex (c and g respectively) showed no staining despite the presence of FITC-vWf positive vessels (d and h).

These experiments further confirmed the exquisite tissue and species specificity of the 3.1 phage. Most importantly, they confirmed that free peptide itself is functional in specifically homing to the synovium and capable of competitively inhibiting the binding of the parent 3.1 phage to the graft MVE ligand.

As a further exercise, the peptide sequences shown above in FIG. 5 were aligned on the basis of the chemical nature (non-polar/hydrophobic, uncharged polar, basic or acidic) of the functional groups of their component amino acids. The following results were obtained:

RLP triple motif related sequences

CHPRLPFAC
CAPNWRLPC

i.e. C-RLP-C

SPS triple motif related sequences

CSPSPFRAC
CSPSRFDQC
CVSPSRTTC

i.e. C-SPSRF-C

HSS triple motif related sequences

CPLSSAQRC
CTWSATSTC
CTHSSATQC
CHTHSSNLC
CPNHSSPHC
CADHSSRHC
CSDYSSRSC

i.e. C-(T/D)HSS(A/R)(T/H)-C

NQR triple motif related sequences

CQTHNQRYC
CTNQRLALC

i.e. C-NQR-C

DRL triple motif related sequences

CKSTHDRLC
CPFHDRHSC
CHPSDRLSC
CDRLNHQFC

i.e. C-HDRL-C

Wherein C-, -C represent any type or number of amino acids preceding or following, respectively, the motif within the flanking cysteines.

Discussion

The isolation of homing peptides specific for human synovium by in vivo phage display selection has been described herein. Homing peptides were identified by sequencing of the peptide-encoding DNA inserts contained by phage preferentially localising to human synovial tissue transplanted into SCID mice.

Such synovial homing phage were isolated by multiple cycles of enrichment in animals transplanted either with synovium only or synovium and skin tissue. This latter study design allowed the pep-PDL to localise, at each round of selection, to either human tissue. Thus the skin grafts could act both as ‘sinks’ to absorb phage recognising common human vascular determinants and as controls for tissue specificity. After three rounds of selection, the inventors observed a significant enrichment of phage localising to synovial grafts but not to skin control grafts. Similarly, despite the considerable circulatory volume passing through the kidneys no enrichment was seen in this mouse tissue. It is also worth noting that the number of phage recovered from the mouse kidneys was similar to that of the skin grafts suggesting that this is likely to represent the background level of binding. Furthermore, the strep-clone-1 control phage, at the same concentration, showed a similar low level of localisation to all three organs. These observations were confirmed and extended to a fourth round of in vivo selection, in a different set of experiments, using animals transplanted with human synovium alone. In the fourth round, the enrichment was over 600 fold greater than in the first round. Therefore, these experiments confirmed the feasibility of using the human/SCID mouse transplantation model to select in vivo phage that preferentially localise to synovial grafts in preference to other human (skin) or mouse (kidney) tissues. This is similar to what has been reported using a ‘pure’ mouse system (28). The great advantage of the model described herein, of course, is that it allows the selection of phage with homing specificity for human tissue determinants presented by the grafts.

The synovial homing specificity of the selected phage was then re-examined in recirculation studies in vivo using SCID animals again double transplanted but with skin and synovium obtained from a patient affected with OA rather than RA. Phage clones recovered from RA synovial grafts, after the third and fourth round of in vivo selection, homed back preferentially to OA synovial grafts, while the control phage showed a modest synovial localisation comparable to the base-line level of the control skin tissue. These experiments provided robust evidence in support of the tissue specificity of the isolated synovial homing phage in several ways. First, they proved that the homing specificity is a stable feature (over time and in different experiments) of such phage. Second, they confirmed that the preferential synovial localisation is independent of the disease status (RA vs OA) of the original transplanted tissue and may suggest that organ specificity is ontogenetically determined. Third, on the basis of the consistent pattern of behaviour of the strep-clone-1 control phage in comparison to synovial peptide phage, they strongly indicated that the specific homing properties were mediated by the peptides themselves rather than the phage component.

To further investigate this aspect, the inventors carried out sequence analysis of the peptide-encoding DNA inserts of 90 randomly selected phage clones from the phage pools recovered from the synovial grafts. This revealed an enrichment of specific sequences. Alignment of the obtained sequences identified several triple and quadruple peptide consensus motifs. Some of the triple peptide motifs were shared and/or overlapped in more than one clone, with some clones possessing more than one motif. In addition, some of the motifs were also found to recur in more than one experiment. Thus, the fact that consensus motifs were found in different phage clones recovered from synovial grafts from different experiments suggests that their occurrence is likely due to a ligand(s)-mediated selection process. Moreover, it also suggests that such motifs are likely to be important in recognizing specific synovial determinants. This was further tested by examining the homing properties of three individual clones (1.23, 2.10 and 3.1) as representatives of frequently occurring motifs. All three clones showed a significantly greater localization to synovial grafts in comparison to strep-clone-1 phage control. In particular clone 3.1 displaying the sequence CKSTHDRLC, containing the DRL consensus motif, showed an increase of approximately 10 fold over the control. Therefore, this sequence was chosen to address the question directly of whether the peptide itself, independently from the displaying phage, retains the property of homing to synovial grafts. The synthetic peptide CKSTHDRLC was shown not only to maintain the synovial homing specificity in vivo but, more importantly, to competitively inhibit the binding of the parent phage to the cognate synovial MVE ligand(s).

From the work presented herein, it can be postulated that the synovial ligand(s) is presented by the MVE as indicated by the intense co-localization of the M13-phage and CKSTHDRLC-peptide immunoreactivity and human MVE within the grafts. Although it is possible that the MVE ligand(s) may be the still elusive synovial specific ‘addressin’ (17), an interesting aspect to consider is that molecules involved in tissue-specific homing have been described that are not classical CAMs. For example, a membrane dipeptidase, particularly accessible in vivo in the lung compared to other tissues, is the receptor for a lung-targeting peptide identified by in vivo phage display (40).

It has also been demonstrated herein that the synovial homing peptides not only are tissue specific (binding to synovial but not skin grafts) but also species specific (binding to human but not mouse tissue). Therefore, it is unlikely that these peptides are binding to a ‘common’ cell adhesion determinant expressed universally by endothelial cells. Equally unlikely is the prospect that the synovial ligand(s) is an inflammation-dependent endothelial CAM, as the inventors have previously demonstrated in the graft vasculature 4 weeks post-transplantation a down-modulation of molecules such as ICAM-1 VCAM-1 and E-Selectin (34). This raises the intriguing possibility that one may be dealing with constitutively expressed determinants possibly involved in a basal recirculation part of the process of immune surveillance. Another possibility is that the synovial ligand(s) represent neo-angiogenic epitopes, given that graft maintenance depends on new blood vessels forming mouse-human anastomoses. However, the synovial homing phage do not bind to control skin grafts, shown to have a similar or slightly higher degree of neo-vascularisation compared to synovial grafts. Thus, to explain the above findings on this basis, it would be necessary to invoke an element of tissue specificity in the phenomenon of synovial neo-angiogenesis as it has been postulated for tumour related vessels (29, 31).

This is the first time that peptides with homing properties specific for human synovial MVE have been reported. This was achieved by a novel approach targeting human tissues, transplanted into SCID mice, directly by in vivo phage display selection. The identification of such peptides, independently of the nature of their ligand(s), opens the possibility of using these sequences to construct joint-specific delivery tools capable of concentrating drugs or gene vectors, directly or via liposomes, specifically to this tissue as has been shown in other systems (31,41,42). Additional experiments using multiple organ transplants, including RA and OA synovium, in the same SCID animals may also be performed to further confirm synovial specificity.

Although the method of the present invention has been described with particular reference to the use of a phage display technique, it is not limited to such a technique and the peptides may alternatively be screened in vivo either on their own or conjugated to a marker molecule. The main advantage of using phage display is simply that it allows recovery of the nucleic acid expressing the peptide in essentially the same step as recovery of the peptide itself. Furthermore, the method may readily be used to identify peptides capable of specific binding to tissues other than synovial tissue. These tissues need not necessarily be human in origin.

A suitable methodology for the grafting of synovial or non-synovial tissues into mice is illustrated by the following example carried out using human peripheral lymph nodes (huPLN).

Tissue collection, preparation, storage & transplantation. Para-aortic or cervical huPLN were obtained from patients requiring vascular surgery. HUPLN were of normal size and macroscopic appearance. Samples of each node were processed for routine H&E histology prior to their use for transplantation studies and found to have a normal histological appearance. Procedures were performed after informed consent approved by the hospital Ethics Committee (LREC n 99/03/19). Samples were divided into two parts. One part was used for immunohistology and the second for transplantation. The part assigned for immunohistology was embedded in Optimal Cutting Temperature compound (OCT, Miles, Calif.), snap frozen in liquid nitrogen cooled isopentane (BDH) and stored at −70° C. until analysis. The second part, assigned for transplantation, was cut into 0.5 cm³ pieces, frozen in 20% DMSO (Sigma) in heat inactivated foetal calf serum (PAA Labs GmbH, Linz, Austria) and stored in liquid nitrogen until engraftment (as described in Wahid et al. (2000). Clin. Exp. Immunol. 122, 133-142). Samples of huPLN were thawed from liquid nitrogen storage immediately before surgery, washed in saline and kept in saline moistened sterile gauze over ice until transplanted. Beige SCID C.B-17 (NOD/LtSz-scid/scid) mice, maintained under pathogen free conditions in biological facilities of Kings College, were anaesthetised by i.p. injection of 0.2 ml Dormitor (0.1 mg/ml SKB) and 0.1 ml ketamine (0.1 mg/ml SKB). A small incision was made in the dorsal skin behind the ear of each SCID mouse (4-6 weeks of age) and the tissue inserted subcutaneously. The wound was closed with soluble suture material (Ethicon). Successful tissue transplantation was assessed prior to migration studies by immunohistology after 4-5 weeks. This particular strain of mice was chosen to minimise this possibility that huPBL could be killed by mouse NK cells in their systemic circulation. NOD/LtSz-scid/scid mice are specifically bred not only to produce no T or B cells, but also to have no NK activity (although the animals retain non-functional NK cells).

Assessment of graft viability. Graft viability was assessed prior to immunohistochemical or morphometric analysis both macroscopically and by microscopy of haematoxylin and eosin stained acetone fixed cryostat sections. Grafts judged to be necrotic or those comprising tissues other than those transplanted (e.g. murine skin and muscle) were excluded from the study.

Assessment of human vasculature within grafts. To confirm the conservation of human vasculature associated cell adhesion molecule (CAM) and to assess the modulation of CAM expression following cytokine/chemokine stimulation of the grafts, the expression of human ICAM1, VCAM1, and E-Selectin were assessed, pre- and post-transplantation using species specific mAb and standard immunohistochemical techniques. The relative expression of CAM's was quantified using an arbitrary scale of staining intensity from 0-4, where 0 indicated no staining and 4 indicated maximal staining. To determine whether the human transplant vasculature remained patent and connected with the murine vasculature infiltrating the grafts, transplanted mice were injected i.v. with either biotinylated anti human ICAM1 or a biotinylated isotype matched control antibody (MOPC21). Mice were killed after 10 minutes and the transplants embedded in OCT and snap frozen. Cryostat sections were then incubated with avidin-biotin-alkaline phosphatase complex (ABC-AP) for 30 minutes followed by development using a Vector Red substrate kit. Sections were subsequently incubated with FITC-conjugated anti human VWFVEi (Serotec, UK), in order to identify human blood vessels and, therefore, determine the site of localisation of the anti ICAM1 and control antibodies. Sections were mounted in aqueous mountant (Immunofluor, ICN Ltd) and examined by UV-fluorescence microscopy.

Reference List

(1) Pitzalis C. Adhesion,migration and cell trafficking. In: Firestein G S, Panayi G S, Wollheim F A, editors. RHEUMATOID ARTHRITIS: New Frontiers in Pathogenesis and Treatment. OXFORD UNIVERSITY PRESS, 2000:137-146.
(2) Firestein GS. Rheumatoid synovitis and pannus. In: Kippel J H, Dieppe P A, editors. Rheumatology. London: Mosby, 1998: 13.
(3) Koch AE. Angiogenesis: Implications for Rheumatoid Arthritis. Arthritis & Rheum 1998; 41(6):951-962.
(4) Haskard DO. Cell adhesion molecules in rheumatoid arthritis. Current Opinion in Rheumatology 1995; 7:229-234.
(5) Pitzalis C. Role of adhesion mechanisms in the pathogenesis of chronic synovitis. (The Michael Mason Prize Essay 1996). B J Rheumatol 1996; 35:1198-1215.
(6) Springer TA. Traffic signals for lymphocyte recirculation and leukocyte emigration:

the multistep paradigm. Cell 1994; 76:301-314.
(7) Baggiolini M. Chemokines and leukocyte traffic. Nature 1998; 392(6676):565-568.
(8) Picker LJ, Butcher EC. Physiological and molecular mechanisms of lymphocyte homing. Ann Rev Immunol 1992; 10:561-591.
(9) Butcher EC, Picker L J. Lymphocyte homing and homeostasis. Science 1996;

272(5258):60-66.
(10) Berg E L, Robinson M K, Warnock R A, Butcher E C. The human peripheral lymph node vascular addressin is a ligand for LECAM-1, the peripheral lymph node homing receptor. J Cell Biol 1991; 114:343-349.
(11) Michie S A, Streeter P R, Bolt P A, Butcher E C, Picker L J. The human peripheral lymph node vascular addressin. An inducible endothelial antigen involved in lymphocyte homing. Am J Pathology 1993; 143:1688-1698.
(12) Berlin C, Berg E L, Briskin M J, Andrew D P, Kilshaw P J, Holzmann B et al. Alpha 4 beta 7 integrin mediates lymphocyte binding to the mucosal vascular addressin MAdCAM-1. Cell 1993; 74:185-195.
(13) Briskin M J, McEvoy L M, Butcher EC. MAdCAM-1 has homology to immunoglobulin and mucin-like adhesion receptors and to IgAl. Nature 1993; 363:461-464.
(14) Campbell J J, Haraldsen G, Pan J, Rottman J, Qin S, Ponath P et al. The chemokine receptor CCR4 in vascular recognition by cutaneous but not intestinal memory T cells. Nature 1999; 400(6746): 776-780.
(15) Kunkel E J, Campbell J J, Haraldsen G, Pan J, Boisvert J, Roberts A I et al. Lymphocyte CC chemokine receptor 9 and epithelial thymus-expressed chemokine (TECK) expression distinguish the small intestinal immune compartment: Epithelial expression of tissue-specific chemokines as an organizing principle in regional immunity. J exp Med 2000; 192(5): 761-768.
(16) Campbell J J, Butcher E C. Chemokines in tissue-specific and microenvironment-specific lymphocyte homing. Curr Opin hnumunol 2000; 12(3): 336-341.
(17) Salmi M, Jalkanen S. How do lymphocytes know where to go: current concepts and enigmas of lymphocyte homing. Advances in Immunology 1997; 64:139-218.
(18) Girard J-P, Springer T A. High endothelial venules (HEVs): specialized endothelium for lymphocyte migration. Immunol Today 1995; 16:449-457.
(19) Augustin H G, Kozian D H, Johnson R C. Differentiation of endothelial cells: analysis of the constitutive and activated endothelial cell phenotypes. Bioessays 1994; 16(12): 901-906.
(20) Borsum T, Hagen I, Henriksen T, Carlander B. Alterations in the protein composition and surface structure of human endothelial cells during growth in primary culture. Atherosclerosis 1982; 44(3):367-378.
(21) de Bono D P, Green C. The adhesion of different cell types to cultured vascular endothelium: effects of culture density and age. Br J Exp Pathol 1984; 65(1): 145-154.
(22) Smith G P, Scott J K. Libraries of peptides and proteins displayed on filamentous phage. Methods Enzymol 1993; 217:228-257.
(23) Sidhu S S, Lowman H B, Cunningham B C, Wells J A. Phage display for selection of novel binding peptides. Methods Enzymol 2000; 328:333-363.
(24) Cwirla S E, Peters E A, Barrett R W, Dower W J. Peptides on phage: a vast library of peptides for identifying ligands. Proc Natl Acad Sci U S A 1990; 87(16): 6378-6382.
(25) Barbas C F, III, Kang A S, Lemer R A, Benkovic S J. Assembly of combinatorial antibody libraries on phage surfaces: the gene III site. Proc Natl Acad Sci U S A 1991; 88(18): 7978-7982.
(26) Winter G, Griffiths A D, Hawkins R E, Hoogenboom H R. Making antibodies by phage display technology. Annu Rev Immunol 1994; 12:433-455.
(27) Nissim A, Hoogenboom H R, Tomlinson I M, Flynn G, Midgley C, Lane D et al. Antibody fragments from a ‘single pot’ phage display library as immunochemical reagents. EMBO J 1994; 13(3):692-698.
(28) Rajotte D, Arap W, Hagedom M, Koivunen E, Pasqualini R, Ruoslahti E. Molecular heterogeneity of the vascular endothelium revealed by In vivo phage display. J Clin Invest 1998; 102:430-437.
(29) Arap W, Pasqualini R, Ruoslahti E. Cancer treatment by targeted drug delivery to tumor vasculature in a mouse model. Science 1998; 279:377-380.
(30) Trepel M, Grifman M, Weitzman M D, Pasqualini R. Molecular adaptors for vascular-targeted adenoviral gene delivery. Hum Gene Ther 2000; 11(14):1971-1981.
(31) Ruoslahti E. Targeting tumor vasculature with homing peptides from phage display. Semin Cancer Biol 2000; 10(6):435-442.
(32) Johns M, George A J, Ritter M A. In vivo selection of sFv from phage display libraries. J Immunol Methods 2000; 239(1-2): 137-151.
(33) Gerlag D M, Borges E, Tak P P, Ellerby H M, Bredesen D E, Pasqualini R et al. Suppression of murine collagen-induced arthritis by targeted apoptosis of synovial neovasculature. Arthritis Res 2001; 3(6): 357-361.
(34) Wahid S, Blades MC, DeLord D, Brown I, Blake G, Yanni Y et al. TNFalpha enhances lymphocyte migration into rheumatoid synovial tissue transplanted into SCID mice. Clin Exp Immunol 2000; 122:133-142.
(35) Blades MC, Manzo A, Ingegnoli F, Taylor P R, Panayi G S, Irjala H et al. Stromal cell derived factor-1 (CXCL12) induces cell migration into human lymph nodes transplanted into SCID mice. J Immunol 2002; (in press).
(36) Howie D, Spencer J, DeLord D, Pitzalis C, Wathen NC, Dogan A et al. Extrathymic T cell differentiation in the human intestine early in life. J Immunol 1998; 161(11): 5862-5872.
(37) Kates S A, Sole N A, Beyermann M, Barany G, Albericio F. Optimized preparation of deca(L-alanyl)-L-valinamide by 9- fluorenylmethyloxycarbonyl (Fmoc) solid-phase synthesis on polyethylene glycol-polystyrene (PEG-PS) graft supports, with 1,8-diazobicyclo [5.4.0]-undec-7-ene (DBU) deprotection. Pept Res 1996; 9(3): 106-113.
(38) Pitzalis C, Cauli A, Pipitone N, Smith C, Barker J, Marchesoni A et al. Cutaneous lymphocyte antigen-positive T lymphocytes preferentially migrate to the skin but not to the joint in psoratic arthritis. Arthritis & Rheum 1996; 39:137-145.
(39) Blades M C, Ingegnoli F, Wheller S K, Manzo A, Wahid S, Panayi G S et al. Stromal cell-derived factor 1 (CXCL12) induces monocyte migration into human synovium transplanted onto SCID Mice. Arthritis & Rheum 2002; 46(3): 824-836.
(40) Rajotte D, Ruoslahti E. Membrane dipeptidase is the receptor for a lung-targeting peptide identified by in vivo phage display. J Biol Chem 1999; 274(17): 11593-11598.
(41) George A J. Application of phage display technology to endothelial cell immunobiology. Dis Markers 2000; 16(1,2): 67.
(42) Hart S L, Arancibia-Carcarno C V, Wolfert M A, Mailhos C, O'Reilly N J, Ali R R et al. Lipid-mediated enhancement of transfection by a nonviral integrin- targeting vector. Hum Gene Ther 1998; 9(4): 575-585.

1. A synovial tissue binding peptide comprising an amino acid sequence motif comprising RLP, SPS, HSS, LSS, TWS, YSS, NQR, DRL or DRH.

2. A peptide according to claim 1 in which the motif comprises SPSRF.

3. A peptide according to claim 1 in which the motif comprises (T or D) HSS (A or R) (T or R).

4. A peptide according to claim 1 in which the motif comprises HDRL.

5. A peptide according to claim 1 in which the motif comprises HPRLPFA.

6. A peptide according claim 1 wherein the motif includes a pair of amino acids capable of causing intramolecular cyclization of the peptide.

7. A peptide according to claim 6 wherein the pair is C and C, C and M or M and M.

8. A peptide according to claim 7 wherein the motif is CHPRLPFAC.

9. A peptide according to claim 7 wherein the motif is CKSTHDRLC.

10. A peptide according to claim 6 wherein the motif is cyclized.

11. A peptide consisting of an amino acid sequence motif according to claim 1.

12. A peptide according to claim 1 coupled to a pharmacological or diagnostic agent.

13. A peptide according to claim 12 wherein the pharmacological agent is an anti-inflammatory, cytostatic, cytotoxic or immunosuppressive compound.

14. A peptide according to claim 12 wherein the pharmacological agent is a gene.

15. A peptide according to claim 12 wherein the diagnostic agent is suitable for use in diagnostic imaging.

16. A peptide according to claim 1 for use in therapy.

17. The use of a peptide according to claim 1 in the preparation of a medicament for the treatment of prevention of inflammatory and/or degenerative arthropathies.

18. A pharmaceutical or diagnostic composition comprising a peptide according to claim 1.

19. A composition according to claim 18 formulated as liposomes.

20. A composition according to claim 19 wherein the peptide is present at least on the exterior surface of the liposomes.

21. A nucleic acid sequence coding for a peptide according to claim 1.

22. An antibody or fragment thereof capable of binding to a peptide according to claim 1.

23. A method of identifying peptides capable of binding to a tissue originally from a first mammalian species, the method comprising the steps of:
i) grafting the tissue originating from the first mammalian species into a subject of a second species having an attenuated immunological response;
ii) introducing a plurality of peptides into the second species; and
iii) determining the localization of the peptides within the second species.

24. A method according to claim 23 wherein the peptides are introduced into the species in
the form of fusion proteins with a coat protein of a bacteriophage.

25. A method according to claim 24 wherein the bacteriophage is M13 phase.

26. A method according to claim 25 wherein the coat protein is pIlI.

27. A method according to claim 23 wherein the peptides are flanked by a pair of amino acids capable of causing intramolecular cyclization of the peptides.

28. A method according to claim 27 wherein the pair is C and C, C and M or M and M.

29. A method according to claim 23 wherein the peptides are generated by random in vitro synthesis.

30. A method according to claim 24 wherein the peptides are generated by replication of the bacteriophage, nucleic acid sequences encoding the peptides having previously been inserted into the bacteriophage genome.

31. A method according to claim 23 wherein the first mammalian species is a human.

32. A method according to claim 23 wherein the tissue comprises synovial tissue.

33. A method according to claim 23 wherein the second species is a mouse.

34. A method according to claim 23 wherein the subject of the second species has severe combined immunodeficiency disease.

35. One or more of peptides listed below: CKSTHDRLC, CHPRLPFAC, HPRLPFA, HDRL, (T or D) HSS (A or R) (T or R) SPSRF, RLP, SPS, HSS, LSS, TWS, YSS, NOR, DRL or DRH.

36. A peptide having one of the sequences listed below: CDRLNHQFC, CKSTHDRLC, and CTHSSATQC.

37. A peptide having one of the sequences selected from the group consisting of CKSTHDRLC, CHPRLPFAC, HPRLPFA, HDRL, (T or D) HSS (A or R) (T or R), SPSRF, RLP, SPS, HSS, LSS, TWS, YSS, NOR, DRL, DRH, CDRLNHQFC, CKSTHDRLC, and CTHSSATQC for use for the treatment of inflammatory and degenerative arthropathies.

38. A peptide according to claim 35 coupled to a cytotoxic drug or gene.

39. A peptide according to claim 35 in a liposome formulation.

40. A peptide according to claim 35 conjugated to an imaging modality.

41. A pharmaceutical or diagnostic composition containing a peptide according to claim 35.

42. A pharmaceutical composition according to claim 41, for intravenous administration.

43. A pharmaceutical composition according to claim 42, comprising 0.5 to 5 mg/Kg body weight by intravenous administration.

44. A method of treatment of inflammatory arthritides (including rheumatoid arthritis, psoriatic arthritis sero-negative arthropathies) which comprises administering one or more of the peptides selected from the group consisting of CKSTHDRLC, CHPRLPFAC, HPRLPFA, HDRL, (T or D) HSS (A or R) (T or R), SPSRF, RLP, SPS, HSS, LSS, TWS, YSS, NQR, DRL, DRH, CDRLNHQFC, CKSTHDRLC, and CTHSSATQC.


SEQUENCE LISTING (46 sequences)

SEQ ID NO: 1 - protein, 5 aa, ARTIFICIAL SEQUENCE
  DESIGNED MOTIF
SPSRF

SEQ ID NO: 2 - protein, 6 aa, ARTIFICIAL SEQUENCE
  DESIGNED MOTIF
XHSSXX

SEQ ID NO: 3 - protein, 4 aa, ARTIFICIAL SEQUENCE
  DESIGNED MOTIF
HDRL

SEQ ID NO: 4 - protein, 7 aa, ARTIFICIAL SEQUENCE
  DESIGNED MOTIF
HPRLPFA

SEQ ID NO: 5 - protein, 7 aa, ARTIFICIAL SEQUENCE
  DESIGNED MOTIF
APNWRLP

SEQ ID NO: 6 - protein, 7 aa, ARTIFICIAL SEQUENCE
  DESIGNED MOTIF
SPSPFRA

SEQ ID NO: 7 - protein, 7 aa, ARTIFICIAL SEQUENCE
  DESIGNED MOTIF
SPSRFDQ

SEQ ID NO: 8 - protein, 7 aa, ARTIFICIAL SEQUENCE
  DESIGNED MOTIF
VSPSRTT

SEQ ID NO: 9 - protein, 7 aa, ARTIFICIAL SEQUENCE
  DESIGNED MOTIF
PLSSAQR

SEQ ID NO: 10 - protein, 7 aa, ARTIFICIAL SEQUENCE
  DESIGNED MOTIF
TWSATST

SEQ ID NO: 11 - protein, 7 aa, ARTIFICIAL SEQUENCE
  DESIGNED MOTIF
THSSATQ

SEQ ID NO: 12 - protein, 7 aa, ARTIFICIAL SEQUENCE
  DESIGNED MOTIF
HTHSSNL

SEQ ID NO: 13 - protein, 7 aa, ARTIFICIAL SEQUENCE
  DESIGNED MOTIF
PNHSSPH

SEQ ID NO: 14 - protein, 7 aa, ARTIFICIAL SEQUENCE
  DESIGNED MOTIF
ADHSSRH

SEQ ID NO: 15 - protein, 7 aa, ARTIFICIAL SEQUENCE
  DESIGNED MOTIF
SDYSSRS

SEQ ID NO: 16 - protein, 7 aa, ARTIFICIAL SEQUENCE
  DESIGNED MOTIF
QTHNQRY

SEQ ID NO: 17 - protein, 7 aa, ARTIFICIAL SEQUENCE
  DESIGNED MOTIF
TNQRLAI

SEQ ID NO: 18 - protein, 7 aa, ARTIFICIAL SEQUENCE
  DESIGNED MOTIF
KSTHDRL

SEQ ID NO: 19 - protein, 7 aa, ARTIFICIAL SEQUENCE
  DESIGNED MOTIF
PFHDRHS

SEQ ID NO: 20 - protein, 7 aa, ARTIFICIAL SEQUENCE
  DESIGNED MOTIF
HPSDRLS

SEQ ID NO: 21 - protein, 7 aa, ARTIFICIAL SEQUENCE
  DESIGNED MOTIF
DRLNHQF

SEQ ID NO: 22 - protein, 9 aa, ARTIFICIAL SEQUENCE
  CYCLISED DESIGNED MOTIF
CHPRLPFAC

SEQ ID NO: 23 - protein, 9 aa, ARTIFICIAL SEQUENCE
  CYCLISED DESIGNED MOTIF
CAPNWRLPC

SEQ ID NO: 24 - protein, 9 aa, ARTIFICIAL SEQUENCE
  CYCLISED DESIGNED MOTIF
CSPSPFRAC

SEQ ID NO: 25 - protein, 9 aa, ARTIFICIAL SEQUENCE
  CYCLISED DESIGNED MOTIF
CSPSRFDQC

SEQ ID NO: 26 - protein, 9 aa, ARTIFICIAL SEQUENCE
  CYCLISED DESIGNED MOTIF
CVSPSRTTC

SEQ ID NO: 27 - protein, 9 aa, ARTIFICIAL SEQUENCE
  CYCLISED DESIGNED MOTIF
CPLSSAQRC

SEQ ID NO: 28 - protein, 9 aa, ARTIFICIAL SEQUENCE
  CYCLISED DESIGNED MOTIF
CTWSATSTC

SEQ ID NO: 29 - protein, 9 aa, ARTIFICIAL SEQUENCE
  CYCLISED DESIGNED MOTIF
CTHSSATQC

SEQ ID NO: 30 - protein, 9 aa, ARTIFICIAL SEQUENCE
  CYCLISED DESIGNED MOTIF
CHTHSSNLC

SEQ ID NO: 31 - protein, 9 aa, ARTIFICIAL SEQUENCE
  CYCLISED DESIGNED MOTIF
CPNHSSPHC

SEQ ID NO: 32 - protein, 9 aa, ARTIFICIAL SEQUENCE
  CYCLISED DESIGNED MOTIF
CADHSSRHC

SEQ ID NO: 33 - protein, 9 aa, ARTIFICIAL SEQUENCE
  CYCLISED DESIGNED MOTIF
CSDYSSRSC

SEQ ID NO: 34 - protein, 9 aa, ARTIFICIAL SEQUENCE
  CYCLISED DESIGNED MOTIF
CQTHNQRYC

SEQ ID NO: 35 - protein, 9 aa, ARTIFICIAL SEQUENCE
  CYCLISED DESIGNED MOTIF
CTNQRLAIC

SEQ ID NO: 36 - protein, 9 aa, ARTIFICIAL SEQUENCE
  CYCLISED DESIGNED MOTIF
CKSTHDRLC

SEQ ID NO: 37 - protein, 9 aa, ARTIFICIAL SEQUENCE
  CYCLISED DESIGNED MOTIF
CPFHDRHSC

SEQ ID NO: 38 - protein, 9 aa, ARTIFICIAL SEQUENCE
  CYCLISED DESIGNED MOTIF
CHPSDRLSC

SEQ ID NO: 39 - protein, 9 aa, ARTIFICIAL SEQUENCE
  CYCLISED DESIGNED MOTIF
CDRLNHQFC

SEQ ID NO: 40 - protein, 9 aa, ARTIFICIAL SEQUENCE
  SYNTHETIC PEPTIDE
CGTWSHPQC

SEQ ID NO: 41 - protein, 7 aa, ARTIFICIAL SEQUENCE
  SYNTHETIC PEPTIDE
GXXXHPQ

SEQ ID NO: 42 - protein, 5 aa, ARTIFICIAL SEQUENCE
  PEPTIDE MOTIF
CRLPC

SEQ ID NO: 43 - protein, 7 aa, ARTIFICIAL SEQUENCE
  PEPTIDE MOTIF
CSPSRFC

SEQ ID NO: 44 - protein, 8 aa, ARTIFICIAL SEQUENCE
  PEPTIDE MOTIF
CXHSSXXC

SEQ ID NO: 45 - protein, 5 aa, ARTIFICIAL SEQUENCE
  PEPTIDE MOTIF
CNQRC

SEQ ID NO: 46 - protein, 6 aa, ARTIFICIAL SEQUENCE
  PEPTIDE MOTIF
CHDRLC